(19) United States
(12) Patent Application Publication
(10) Pub. No.: US 2024/0165137 A1
(43) Pub. Date: May 23, 2024

(54) METHODS FOR IMPROVING MUSCLE STRENGTH AND MOBILITY

(71) Applicant: Intrinsic Medicine, Inc., Seattle, WA (US)

(72) Inventors: Alexander Martinez, Des Moines, WA (US); Jason Ferrone, San Diego, CA (US)

(21) Appl. No.: 18/200,747

(22) Filed: May 23, 2023

Related U.S. Application Data
(63) Continuation of application No. PCT/US2021/060251, filed on Nov. 22, 2021.
(60) Provisional application No. 63/118,034, filed on Nov. 25, 2020.

(51) Int. Cl. A61K 31/702

(52) U.S. Cl. CPC A61K 31/702

(57) ABSTRACT

Described are methods of improving muscle strength and mobility comprising administration of a fucosylated oligosaccharide.

### RELATED APPLICATIONS

This application is a continuation of International Application No. PCT/US21/60251, which designated the United States and was filed on Nov. 22, 2021, published in English which claims the benefit of U.S. Provisional Application No. 63/118,034, filed Nov. 25, 2020. The entire contents of the above-referenced applications are incorporated by reference herein.

### BACKGROUND OF THE INVENTION

Loss of muscle mass and strength is a well-known condition of aging. Frailty is a clinical syndrome characterized by increased vulnerability and a decline in reserve and function such that the ability to recover from stressors is impaired (Cadore et al. (2013), Rejuvenation Res. 16(2): 105-114; Harries et al. (2012), Aging Cell. 11(2): 262-268. Frailty affects 7 to 12% of people over 65 years of age and the risk of frailty increases with age. While frailty is associated with aging, the syndrome is also related to lifestyle, chronic disease, and a combination thereof. Frailty is also observed in younger patients with chronic health conditions (Richards et al. (2019), PLoS One 14(7): e0219083). Other diseases and conditions, such as malnutrition, cancer, cardiovascular disease, and inflammatory disease, can exacerbate the effects of frailty syndrome.

One feature of frailty syndrome is severe muscle loss, or sarcopenia. Age-related sarcopenia is considered “primary sarcopenia.” “Secondary sarcopenia” can result from various diseases and conditions including diabetes mellitus and chronic obstructive pulmonary disease (Morley 2018, J Cachexia Sarcopenia Muscle 9(7): 1196-1199).

Repair of muscle after injury involves an inflammatory response and accumulation of anti-inflammatory/M2 macrophages followed by myoblast differentiation and the formation of new muscle fibers. It has been reported that CCAAT-enhancer binding protein beta (CEBPB) affects these muscle repair process and more specifically, that increased CEBPB expression is associated with higher muscle strength and improved physical performance in human patients (Harries et al. (2012)).

Despite the prevalence of frailty and sarcopenia, there remains a need for effective treatments.

### SUMMARY OF THE INVENTION

The present invention is based, at least partially, on the discovery that the fucosylated non-digestible oligosaccharide, 2′-fucosyllactose, upregulates CEBPB expression in LPS-stimulated THP-1 cells.

The invention therefore encompasses a method of improving muscle strength or mobility in a subject in need thereof comprising administering to said subject an effective amount of a fucosylated oligosaccharide, such as a fucosylated non-digestible oligosaccharide. The invention also encompasses a method of increasing CEPBP expression in a subject in need thereof comprising administering to said subject an effective amount of a fucosylated oligosaccharide, such as a fucosylated non-digestible oligosaccharide. The invention additionally encompasses methods of reducing the risk of impaired mobility, muscle loss and/or muscle weakness comprising administering to the subject an effective amount of a fucosylated oligosaccharide, such as a fucosylated non-digestible oligosaccharide. The fucosylated oligosaccharide can be selected from the group comprising 2′-fucosyllactose (2′-FL), 3-fucosyllactose (3′-FL), difucosyllactose, lacto-N-fucopentaoses (that is to say lacto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. In certain aspects, the fucosylated non-digestible oligosaccharide is 2′-fucosyllactose (2′-FL).

The subject to be treated can be a human subject, for example. The human subject can be a child or an adult. In certain aspects, the subject is over 50 years old, over 60 years old, over 65 years old, over 70 years old, over 75 years old, over 80 years old, or over 85 years old. In further aspects, the subject is suffering from low muscle mass or impaired physical mobility. In additional aspects, the subject is suffering from and/or diagnosed with and/or at risk of a condition selected from the group consisting of frailty or frailty syndrome, pre-frailty, sarcopenia, age-related muscle loss, and a condition resulting in muscle atrophy and/or fatigue. For example, the age-related muscle loss can be at least partially due to diabetes or obesity and/or the condition resulting in muscle atrophy and/or fatigue are selected from the group consisting of intensive care unit-induced skeletal muscle weakness, cancer cachexia-induced skeletal muscle weakness or fatigue, and muscle weakness in chronic inflammatory disease; and/or the impaired mobility is due to aging, stroke, rheumatoid arthritis, osteoarthritis, osteoporosis, osteopenia, limb fracture, multiple sclerosis, trauma, sepsis, or obesity. In further aspects, the subject is suffering from at risk of developing drug-induced myopathy or drug-induced muscle weakness, muscle weakness associated with infection; muscle weakness associated with an endocrine condition, an inflammatory condition, a rheumatologic condition, an electrolyte syndrome, a neuromuscular condition, a neurological condition, or a genetic condition.

In certain aspects, the subject that is treated has reduced CEBPB expression (for example, as determined by measuring RNA) as compared to that of a control expression level prior to the initiation of the treatment. The method can, for example, comprise obtaining a biological sample from the subject prior to the initiation of treatment, detecting the expression level of CEBPB in the biological sample, and administering the fucosylated oligosaccharide to a subject determined to have an expression level of CEBPB lower than that of a control expression level.

In yet further aspect, the expression level of CEBPB increases after the initiation of the therapy. The expression level of CEBPB can, for example, increase by an amount of about 10% or more, 20% or more, 30% or more, 40% or more, 50% or more, or 60% or more as compared prior to the initiation of the treatment. In additional aspects, the treatment improves the subject's muscle strength or mobility as measured by lower handgrip muscle strength, short physical performance battery (SPPB) score, Timed Up and Go test (TUGT), walking speed (WS), and/or grip strength (GS) (described, for example, in Harries et al. (2012); and Phu et al. (2020), BMC Geriatr 20: 242; the contents of each of which are expressly incorporated by reference herein).

The fucosylated oligosaccharide can be administered orally, for example. The fucosylated nondigestible oligosaccharide can be administered in an amount of at least about 145 mg/L and/or in an amount from about 1 g to about 20 g per day.

The fucosylated oligosaccharide can be administered in a pharmaceutical composition comprising a pharmaceutically acceptable carrier or excipients, wherein the pharmaceutical composition is not mammalian milk or is not human milk. In certain aspects, the composition comprises at least about 9% by weight(e.g., about 10, 15, 20, 25, 30, 35, 40, 45, 50, 55, 60, 65, 70, 75, 80, 85, 90, 95, or 100%; or any value between any of the foregoing) of the fucosylated nondigestible oligosaccharide. The pharmaceutical composition can be formulated as a tablet, capsule, or a powder, for example.

### DETAILED DESCRIPTION OF THE INVENTION

As used herein and in the appended claims, the singular forms “a,” “an,” and “the” include plural referents unless the context clearly dictates otherwise. Thus, for example, reference to “an oligosaccharide” includes a plurality of such oligosaccharides and reference to “the therapeutic agent” includes reference to one or more therapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs.

Although many methods and reagents are similar or equivalent to those described herein, the exemplary methods and materials are disclosed herein. All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like. The term “about” as used herein, in reference to a numerical value or range, allows for a degree of variability in the value or range, for example, within 10%, within 5%, or within 4%, or within 2% of the value or range.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “release controlling excipient” as used herein, refers to an excipient whose primary function is to modify the duration or place of release of the active substance from a dosage form as compared with a conventional immediate release dosage form.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient. In certain preferred aspects, the subject is a human patient.

The term “substantially pure” as used herein in reference to a given oligosaccharide means that the oligosaccharide is substantially free from other biological macromolecules. The substantially pure oligosaccharide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The terms “treat”, “treating” and “treatment,” as used herein, refers to ameliorating symptoms associated with a disease or disorder, including preventing or delaying the onset of the disease or disorder symptoms, and/or lessening the severity or frequency of symptoms of the disease or disorder.

An “effective amount” or a “therapeutically effective amount” of a fucosylated oligosaccharide as described herein refers to an amount of the compound that is sufficient to achieve a specific effect or result, and/or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. In certain aspects, an effective amount is an amount that increases expression of CEBPB. The term “effective amount” can also refer to an amount of the oligosaccharide that improves muscle strength and/or improves physical mobility of the subject as described herein. The term “effective amount” can additionally refer to an amount of the oligosaccharide that slows or inhibits progression and worsening of symptoms, such as impaired mobility, as compared to that in the absence of the treatment.

The terms “active ingredient” and “active substance” refer to an oligosaccharide or compound or agent, which is administered, alone or in combination with one or more pharmaceutically acceptable excipients and/or carriers, to a subject for treating, preventing, or ameliorating one or more symptoms of a disorder.

The terms “drug,” or “therapeutic agent,” refer to a compound, or a pharmaceutical composition thereof, which is administered to a subject for treating, preventing, or ameliorating one or more symptoms of a disorder.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms.

Fucosylated oligosaccharides are a class of human milk oligosaccharides (HMOs) that have been associated with the production of anti-inflammatory short-chain fatty acids. Fucosylated oligosaccharides include, for example, 2′-fucosyllactose, 3-fucosyllactose, difucosyllactose, lacto-N-fucopentaoses (that is to say lacto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. In certain aspects, the fucosylated non-digestible oligosaccharide is 2′-fucosyllactose (2′-FL). In certain aspects, the fucosylated oligosaccharide is 2′-fucosyllactose (2′-FL), 3-fucosyllactose (3′-FL), difucosyllactose (DFL). In yet further aspects, the fucosylated oligosaccharide is 2′-FL. As used herein, a “fucosylated oligosaccharide” is an oligosaccharide having the three sugar unit backbone, wherein each of the sugar units (fucose (Fuc), galactose (Gal), and glucose (Glc)) can be independently either in its native form or in a modified form. For example, the modified form of a sugar unit can be a sugar unit, in which at least one or more (e.g., 1, 2, 3, or more) of the hydroxyl groups is replaced with hydrogen, alkyl or a functional group; such as, for example, hydrogen, substituted or unsubstituted C1-C6 alkyl (e.g., methyl, ethyl), or substituted or unsubstituted amine group.

Fucosyllactose (FL) is a fucosylated non-digestible oligosaccharide present in human milk but not in cow milk. The primary fucosylated HMO is 2′-fuscosyllactose or 2′FL. It consists of three monosaccharide units, fucose, galactose and glucose linked together. Lactose is a galactose unit linked to a glucose unit via a beta 1,4 linkage. A fucose unit is linked to a galactose unit of a lactose molecule via an alpha 1,2 linkage (2′-fucosyllactose, 2′-FL) or via an alpha 1,3 linkage to the glucose unit of a lactose (3-Fucosyllactose, 3-FL). 2-′FL has the chemical structure shown below:

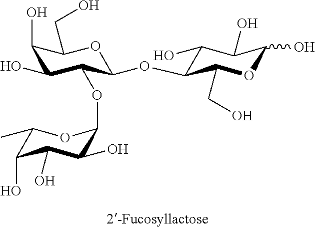

The terms 2′-fucosyllactose or “2′-FL” and “2′FL” are used interchangeably herein. 2′-fucosyllactose has been granted generally regarded as safe (GRAS) status in the U.S. and is regarded by the Europe Food Safety Authority as safe for infant and follow-on formula. 2′-FL has been shown to have many beneficial properties, such as improving of gut health through modulation of the gut microbiome as well as reduction of local gut inflammation in models of necrotizing enterocolitis and other inflammatory bowel diseases. In addition, 2′-FL has been shown to have positive effects on gut epithelial barrier function and also independent anti-inflammatory effects through the reduction in TNFα and IL-8.

In certain aspects, the fucosylated oligosaccharide has the Formula 1A or 1B:

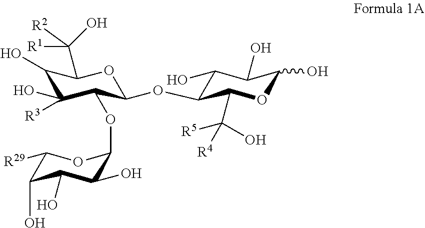

or a pharmaceutically acceptable salt, solvate, or prodrug thereof, wherein:
R¹-R⁵ are each independently selected from H, D, a halo, an unsubstituted or substituted (C₁-C₆)alkyl, an unsubstituted or substituted (C₁-C₆)heteroalkyl, an un substituted or substituted (C₂-C₆)alkenyl, an unsubstituted or substituted (C₂-C₆)heteroalkenyl, an unsubstituted or substituted (C₃-C₆)alkynyl, an unsubstituted or substituted (C₃-C₆) heteroalkynyl, an unsubstituted or substituted (C₄-C₈)cycloalkyl, an unsubstituted or substituted heterocycle, an unsubstituted or substituted aryl, —ROR′, —RN(R′)₂, —RSSR′, —SH, RSOR′, —RSO₂R′, —RSO₂H, —RSO₃H, —RC(═S)—R′, —ROH, —RC(═O)R′, —RNO₂, —RSR′, —RCN, —RNC, —RNNR′, —RC(═O)OR′, —ROC(═O)R′, —RC(═O)H, —RC(═O)OH, —RC(═O)N(R′)₂, —RN₃, —ROCN, —RNCO, —RONO₂, —RNO, —ROP(═O)(OH)₂, and —RB(OH)₂;
R is absent or a (C₁-C₅)alkyl;
R′ is independently selected from H, D, an unsubstituted or substituted (C₁-C₆) alkyl, an unsubstituted or substituted (C₁-C₆)heteroalkyl, an unsubstituted or substituted (C₂-C₆) alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, and an unsubstituted or substituted aryl; and
R²⁹ is an unsubstituted or substituted (C₁-C₆)alkyl.

In yet additional aspects, the fucosylated oligosaccharide has the Formula IIIb:

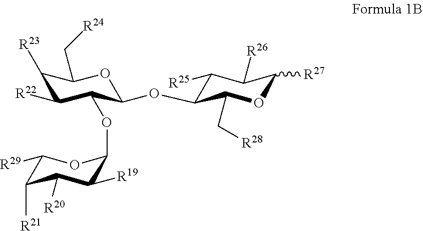

wherein:
one, two or three of R¹⁹-R²⁸ are each independently selected from the group consisting of hydrogen, an unsubstituted or substituted C₁-C₆ alkyl (including, but not limited to, methyl and ethyl) and N(R′)₂ (wherein R′ is as defined above), the remainder or R¹⁹-R²⁸ are —OH, and R²⁹ is substituted or unsubstituted C₁-C₆ alkyl;
or alternatively, one, two or three of R¹⁹-R²⁹ are each independently selected from NHC(O)R″, wherein R″ is unsubstituted or substituted (C₁-C₆) alkyl (including, but not limited to, methyl), the remainder or R¹⁹-R²⁸ are —OH, and R²⁹ is substituted or unsubstituted C₁-C₆ alkyl. In certain aspects, R²⁶ is NHC(O)CH₃ and R¹⁹-R²⁵ and R²⁷-R²⁸ are —OH, and R²⁹ is methyl.

The term “alkyl” refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkenyl” refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. In certain instances, the alkenyl group will be conjugated, in other cases an alkenyl group will not be conjugated, and yet other cases the alkenyl group may have stretches of conjugation and stretches of non-conjugation.

Additionally, if there is more than 2 carbons, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise. The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond.

Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and 0.

Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed there from.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “optionally substituted” means independent replacement of one or more hydrogen atoms with a substituent. The term “optionally substituted” also refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

The methods described herein can be used to improve muscle strength and mobility and/or to increase CEBPB expression in a subject in need thereof comprising administering an effective amount of a fucosylated oligosaccharide as described herein. The preferred fucosylated oligosaccharide is 2′-FL. The effective amount can, for example, be an amount that increases the expression of CEBPB in the subject. The subject to be treated, including, for example, the human subject to be treated can be suffering from impaired mobility and/or muscle loss. In certain aspects, the subject is an elderly adult, for example, an adult about 65 years old or greater. In other aspects, the subject is about 50 years or older, about 60 years or older, about 65 years or older, about 70 years or older, about 75 years or older, about 80 years or older, or about 85 years or older.

Harries et al. (Harries et al. (2012). Aging Cell. 11(2): 262-268; the contents of which are expressly incorporated by reference herein) found that CEBPB expression levels (in circulating leukocytes) were associated with muscle strength in human adults. Specifically, Harries used peripheral blood RNA samples from study participants and the Short Physical Performance Batter score (SPPB) to show that increased CEBPB expression in circulating leukocyte RNA samples is associated with greater muscle strength and physical performance. Thus, the subject to be treated can be a subject with reduced CEBPB expression level as compared to a control expression level. CEBPB expression can be assessed, for example, by measuring RNA level in a biological sample obtained from the subject, for example, in a peripheral blood sample. Subjects can, for example, be screened by measuring CEBPB expression prior to the initiation of the therapy and the fucosylated oligosaccharide can be administered to the subject as described herein if the subject is identified has having reduced CEBPB as compared to a control expression level. The control expression level is, for example, the average in the human population, or the expression level of a normal, healthy individual, or the expression level of said subject at a younger age or prior to the onset of their condition/disease/illness or impaired mobility or muscle weakness. The treatment is “initiated” at the time the first dose of the fucosylated oligosaccharide is administered.

Conditions that can be treated according to the methods described herein include, but are not limited to, impaired mobility, frailty or frailty syndrome, pre-frailty, sarcopenia, age-related muscle loss, metabolic syndrome, and a condition resulting in muscle atrophy and/or fatigue. In certain aspects, diabetes or obesity can be a contributing factor to the age-related muscle loss. Exemplary conditions resulting in muscle atrophy and/or fatigue are selected from the group consisting of intensive care unit-induced skeletal muscle weakness, cancer cachexia-induced skeletal muscle weakness or fatigue, and muscle weakness in chronic inflammatory disease. In other aspects, the impaired mobility can be due to a disease or disorder selected from the group consisting of stroke, rheumatoid arthritis, osteoarthritis, osteoporosis, osteopenia, limb fracture, multiple sclerosis, trauma, sepsis, or obesity. The impaired mobility can also be due to aging.

In specific aspects, the methods described herein can be used for the treatment of sarcopenia. “Sarcopenia” is the age-associated loss of muscle mass and functionality (including, but not limited, muscle strength and gait speed). As used herein, “sarcopenia” encompasses both primary and secondary sarcopenia. Sarcopenia can defined by low physical performance (or mobility limitations) indicated by at least one of the following (a) a walking distance of <400 m in the 6 minute walk test (6MWT); (b) a time of >15 min in the 400 m walk test; (c) a short physical performance battery (SPPB) score of ≤8; (d) a gait speed over a 4-m course of ≤1 m/s, preferably <0.8 m/s, more preferably <0.8 m/s or ≤0.0.8 ms but ≥0.0.3 m/s. Sarcopenia can also be defined by the criterion of low muscle mass (or low skeletal muscle mass) indicated by at least one of the following: (a) a appendicular skeletal muscle index (ASMI) of ≤7.26 kg/m² for men or ≤5.5 kg/m² for women, said ASMI being defined as appendicular skeletal muscle mass divided by the square of height; (b) an appendicular lean (body) mass (AL(B)M) of ≤19.75 kg for men or ≤15.02 kg for women; (c) an AL(B)M adjusted for body mass index (BMI) of 0.789 kg for men or ≤0.512 kg for women; said ASMI and AL(B)M being measured by dual energy X-ray absorptiometry (DXA) and said TMV being measured by magnetic resonance imaging (MRI). Sarcopenia can additionally be defined by the criterion of low muscle strength (or weakness) indicated by a value of <30 kg, preferably <26 kg, for men or <20 kg, preferably <16 kg, for women in the handgrip strength test. The invention includes, for example, a method for improving muscle strength and/or reducing the risk of sarcopenia in obese or overweight adults participating in a weight loss program.

In additional aspects, the inventive methods can be used to treat frailty or frailty syndrome. “Frailty” is defined as a clinical syndrome associated with increased vulnerability resulting from a decline in reserve and function across multiple physiologic systems such that the ability to recover from stressors is compromised. Frailty is a syndrome with symptoms such as low body weight due to unintentional weight loss, exhaustion, weakness, slow walking and low physical activity. Frailty is characterized by a decreased reserve and resistance to stressors, in turn resulting from cumulative decline across multiple physiologic systems, and causing a vulnerability to adverse outcomes. Increased insulin resistance, metabolic syndrome and osteoporosis increase among these. Frailty has been defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with VO2 max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year) (Fried et al., “Frailty in older adults: evidence for a phenotype.” J. Gerontol. A. Biol. Sci. Med. Sci. 56(3): M146-M156 (2001)). A consequence of frailty is increased incidence of falls and associated injuries like fractures and impaired mobility. Together with muscle mass related lower immunity, reduced healing rates and mental deterioration, this can lead to a loss of independence.

Pre-frailty or the pre-frail stage is when one or two of the Fried et al. criteria described above are present and identifies an individual at a high risk of progressing to frailty. In certain aspects, the invention is used in the treatment of pre-frailty, for example, in slowing or impeding the progression of the pre-frail subject and/or slowing the progression to frailty.

The method described herein can be used to treat patients that have experienced muscle loss. Such muscle loss can occur with age, for example, age-related muscle loss. The muscle loss or atrophy can also be due to intensive care-induced skeletal muscle weakness, cancer cachexia-induced muscle weakness and fatigue, or muscle weakness in chronic inflammatory diseases (see for example, Kalyani et al. (2014), Lancet Diabetes Endocrinol. 2(10): 819-829 and Powers et al. (2017), Med Sci Sports Exerc. 48(11): 2307-2319. The method can be used for, example, to treat drug-induced myopathy, including, for example, muscle weakness or myopathy induced by alcohol, cocaine, glucocorticoid, lipid-lowering drug (e.g., statin), antimalarial, colchicine, chemotherapeutic agent, amiodarone, anti-thyroid agent (e.g., methimazole or propylthiouracil), anti-retroviral (zidovudine or lamivudine), cimetidine, fibric acid derivatives (e.g., gemfibrozil), interferon, leuprolide acetate, NSAID, penicillin, and sulfonamides. The described methods can additionally be used to treat muscle weakness associated with infection (e.g., limited to, Epstein-Barr virus, HIV, influenza, meningitis, polio, rabies, syphilis, toxoplasmosis, and coronavirus which includes, but is not limited to, human coronaviruses 229E, NL63, OC43, and HKU1, SARS-CoV, MERS-CoV, and SARS-CoV-2/COVID-19), endocrine conditions (e.g., acromegaly, primary hyperthyroidism, hypopituitarism, vitamin D deficiency), inflammatory conditions, rheumatologic conditions (e.g., polymyalgia and systemic sclerosis/scleroderma), electrolyte syndromes (e.g., hypercalcemia, hyperkalemia, hypokalemia, hypermagnesemia, hypomagnesemia), neuromuscular conditions (e.g., amyotrophic lateral sclerosis, muscular dystrophy, myasthenia gravis, spinal muscular atrophy), neurological conditions (cervical spondylosis, Guillain-Barre syndrome, botulism, Lambert-Eaton myasthenic syndrome, multiple sclerosis, spinal cord injury), or a genetic condition (e.g., distal myopathies and oculopharyngeal muscular dystrophy).

The method described herein can also be used to treat a patient at risk of experiencing muscle loss, at risk of impaired mobility, or at risk of muscle weakness. Such methods including reducing the incidence, progression, or worsening of muscle loss, impaired mobility, or muscle weakness. Patients at risk of experiencing muscle loss including elderly patients, hospitalized patients, patients suffering from cancer or chronic inflammatory disease, patients undergoing therapy with a drug known to induce muscle weakness or myopathy (including those specifically described above), subject suffering from a condition associated with muscle weakness or myopathy (including infection, endocrine conditions, inflammatory conditions, electrolyte syndromes, neuromuscular conditions, neurological conditions, and genetic conditions), patients at risk of developing age-related impaired mobility (e.g. elderly subject, hospitalized subjects, subjects suffering from obesity, subjects suffering from diabetes, or a combination thereof).

The fucosylated oligosaccharide can be administered at various intervals, for example, once a day, twice a day, three times a day, once a week, twice a week, or as needed.

The fucosylated oligosaccharide can be administered with an additional active agent, including, but not limited, an additional active agent for improving muscle strength and/or improving mobility. The fucosylated oligosaccharide, can for example, be administered concurrently with the additional active agent, or separately from the additional active agent. The fucosylated oligosaccharide treatment can be incorporated in a treatment regimen that includes exercise, physical therapy, and/or an adequate protein diet.

The fucosylated oligosaccharide (including, but not limited to, 2′-FL) can be administered as part of a pharmaceutical composition, wherein the pharmaceutical composition comprises the fucosylated oligosaccharide and a pharmaceutically acceptable carrier or excipient. In certain aspects, the composition is not mammalian breast milk. In certain aspects, the composition is not human breast milk. In yet further aspects, the composition is not a milk product including, but not limited to, infant formula and toddler's milk.

As discussed above, the fucosylated oligosaccharide, for example, 2′-FL, can be administered as part of a pharmaceutical composition. For example, the pharmaceutical compositions can comprise 2′-FL as the only oligosaccharide in the composition. In additional aspects, the composition can further comprise at least one or more additional (e.g., 1, 2, 3, or more) oligosaccharides, including one or more other human milk oligosaccharides.

In further aspects, the methods additionally provide an improvement in the subject's microbiota (gut and/or oral) composition. Improvement in, or avoidance of, gastrointestinal symptoms, such as constipation, diarrhea, stool consistency, stool smell, flatulence and abdominal pain is desirable, such as, for example, at weeks 8 and 16 of the beginning of treatment.

In certain aspects, the fucosylated oligosaccharide is administered orally. Oral administration of the oligosaccharides of the disclosure provide for systemic circulation of the oligosaccharides both in infants and adults. Unlike other drug products approved by the FDA, the oligosaccharides described herein can not only be administered to treat a disease or disorder in an adult subject, but can also be administered to pregnant females, infants, and subjects who have impaired organ function (e.g., liver disfunction, kidney failure). Due to the oligosaccharides of the disclosure having little to no adverse effects in humans, this form of therapy could be used as a preventive, as a first line therapy option, or as an adjunct to existing therapies that would be well tolerated by patients of either sex.

In a further embodiment, said oligosaccharide is substantially a single enantiomer, a mixture of about 90% or more by weight of the (—)-enantiomer and about 10% or less by weight of the (+)-enantiomer, a mixture of about 90% or more by weight of the (+)-enantiomer and about 10% or less by weight of the (—)-enantiomer, substantially an individual diastereomer, or a mixture of about 90% or more by weight of an individual diastereomer and about 10% or less by weight of any other diastereomer.

The oligosaccharides disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of an oligosaccharide in its (R) form is equivalent, for oligosaccharides that undergo epimerization in vivo, to administration of the oligosaccharide in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the oligosaccharide disclosed herein contains an acidic or basic moiety, it may also be disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (—)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The oligosaccharide as disclosed herein may also be designed as a prodrug, which is a functional derivative of the oligosaccharide as disclosed herein and is readily convertible into the parent oligosaccharide in vivo. Prodrugs are often useful because, in some situations, they may be easier to administer than the parent oligosaccharide. They may, for instance, be bioavailable by oral administration whereas the parent oligosaccharide is not.

The prodrug may also have enhanced solubility in pharmaceutical compositions over the parent oligosaccharide. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72,324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The oligosaccharide may be produced by biotechnological means using enzyme-based fermentation technology (recombinant or natural enzymes) or microbialfermentation technology. In the latter case, microbes may either express their natural enzymes and substrates or may be engineered to produce respective substrates and enzymes. Single microbial cultures and/or mixed cultures may be used. Alternatively, the oligosaccharides may be produced by chemical synthesis from lactose and other substrates.

Biotechnological approaches have made it possible for the large scale, cost-efficient production of target oligosaccharides.

Precisely, the oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO2014135167A1.

Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2t-FL can be made as described in WO 2010/115934 and WO 2010/115935, 3-FL can be made as described in WO 2013/139344. Fucosylated oligosaccharides can be made as described in WO 2012/127410. With regard to biotechnological methods, WO 2001/04341 and WO 2007/101862 describe how to make oligosaccharides optionally substituted by fucose using genetically modified E. coli. The oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO2014135167A1.

The fucosylated oligosaccharide may be isolated by chromatography or filtration technology from a natural source such as animal milks. Alternatively, it may be produced by biotechnological means using specific fucosyltransferases and/or fucosidase either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Alternatively, fucosylated oligosaccharides may be produced by chemical synthesis from lactose and free fucose. Fucosylated oligosaccharides are also available for example from Kyowa Hakko Kogyo of Japan.

The pharmaceutical composition can comprise a fucosylated oligosaccharde or a pharmaceutically acceptable salt, solvate, or prodrug thereof, as an active ingredient, combined with a pharmaceutically acceptable vehicle, carrier, diluent, or excipient, or a mixture thereof; in combination with one or more pharmaceutically acceptable excipients or carriers.

Disclosed herein are pharmaceutical compositions in modified release dosage forms, which comprise one or more oligosaccharides (e.g., 2′FL or derivatives thereof) of the disclosure, or a pharmaceutically acceptable salt, solvate, or prodrug thereof; and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise one or more oligosaccharides (e.g., 2′FL or derivatives thereof) as disclosed herein, or a pharmaceutically acceptable salt, solvate, or prodrug thereof; and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise one or more oligosaccharides (e.g., 2′FL or derivatives thereof) as disclosed herein in substantially pure form (e.g., lacking other oligosaccharides found in milk), or a pharmaceutically acceptable salt, solvate, or prodrug thereof; and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of one or more oligosaccharides (e.g., 2′FL or derivatives thereof) disclosed herein in the form of at least two consecutive pulses separated in time (e.g., separated in time from 0.1 up to 24 hours or a few days). The pharmaceutical compositions comprise an oligosaccharide as disclosed herein, or a pharmaceutically acceptable salt, solvate, or prodrug thereof; and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise one or more oligosaccharides (e.g., 2′FL or derivative thereof) as disclosed herein, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 1000 mg, or up to 1500 mg, or up to 2000 mg, or up to 3000 mg (or any value between 0.1-3000 mg), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of the oligosaccharide as disclosed herein, in the form of immediate release tablets for oral administration. The pharmaceutical compositions further comprise inactive ingredients such as flavoring agents, copovidone, ethylcellulose, magnesium stearate, mannitol, and silicon dioxide.

Provided herein are pharmaceutical compositions that comprise about 0.1 to about 1000 mg or up to 2000 mg or up to 15000 mg (or any value there between), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of the oligosaccharide as disclosed herein, in the form of extended release tablets for oral administration. The pharmaceutical compositions further comprise inactive ingredients such as ethylcellulose, dibutyl sebacate, polyvinyl pyrroliodone, sodium stearyl fumarate, colloidal silicon dioxide, and polyvinyl alcohol.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the oligosaccharide sufficient to produce the desired therapeutic effect, in association with the required pharmaceutical carriers or excipients. Examples of unit-dosage forms include ampoules, syringes, and individually packaged to capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The oligosaccharides as disclosed herein may be administered alone, or in combination with one or more other oligosaccharides disclosed herein, and/or one or more other active ingredients. The pharmaceutical compositions that comprise an oligosaccharide disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms.

These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the oligosaccharides may be administered chronically, that is, for an extended period of time, including throughout the duration of the patient's life in order to ameliorate or otherwise control or limit the symptoms of the patient's disease or condition. Once improvement of the patient's condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The pharmaceutical compositions disclosed herein may be formulated in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastimes, cachets, pellets, medicated chewing gum, granules, bulk powders, effervescent or non-effervescent powders or granules, solutions, emulsions, suspensions, solutions, wafers, sprinkles, elixirs, and syrups. In addition to the oligosaccharides, the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethyl cellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethyl cellulose (HEC), hydroxypropyl cellulose (HPC), hydroxypropyl methyl cellulose (HPMC); microcrystalline celluloses, such as AVICEL-PH-101, AVICEL-PH-103, AVICEL RC-581, AVICEL-PH-105(FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

It should be understood that many carriers and excipients may serve several functions, even within the same formulation. The pharmaceutical compositions disclosed herein may be formulated as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be formulated in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative.

Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol.

The pharmaceutical compositions disclosed herein may be formulated as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions disclosed herein can be formulated as an oral nutritional composition. An oral nutritional composition can contain sources of protein, lipids and/or digestible carbohydrates and can be in solid, powdered or liquid forms. The composition can be designed to be the sole source of nutrition or a nutritional supplement. Suitable protein sources include intact, hydrolyzed, and partially hydrolyzed protein, which can be derived from any suitable source such as milk (e.g., casin, whey), animal (e.g., meat, fish), cereal (e.g., rice, corn), and vegetable (e.g., soy, potato, pea), insect (e.g., locust) and combinations of these sources. Examples of the source of protein include whey protein concentrates, whey protein isolates, whey protein hydrolysates, acid.

In a certain embodiment, the composition described herein can further comprise one or more foodgrade agents. Examples of foodgrade agents that can be used with the oligosaccharides disclosed herein, include, but are not limited to, milk (e.g., cow's milk, almond milk, soy milk), yogurt, maltodextrin, milk protein concentrate, Sucromalt, glycerine, cocoa powder, soy protein isolate, fructose, vegetable or animal oils (e.g., high oleic safflower oil, soy oil, canola oil), plant sterol esters, HMSs/HMOs, soy lecithin, carrageenan, taurine, L-carnitine, vitamins and/or minerals (e.g., sodium ascorbate, potassium citrate, sodium phosphate, calcium citrate, choline chloride, potassium chloride, sodium citrate, magnesium oxide, alpha-tocopheryl acetate, zinc sulfate, ferrous sulfate, niacinamide, calcium pantothenate, vitamin A palmitate, citric acid, manganese sulfate, pyridoxine hydrochloride, vitamin D3, copper sulfate, thiamine mononitrate, riboflavin, beta carotene, folic acid, biotin, potassium iodide, chromium chloride, sodium selenate, sodium molybdate, phytonadione, vitamin B12, magnesium chloride, calcium phosphate).

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action.

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungi static concentrations. All parenteral formulations must be sterile, as known and practiced in the art.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

The pharmaceutical compositions disclosed herein may be administered topically to the skin, orifices, or mucosa. The topical administration, as used herein, include (intra)dermal, conjunctival, intracorneal, intraocular, ophthalmic, auricular, transdermal, nasal, vaginal, ureteral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions disclosed herein may be administered intranasally or by inhalation to the respiratory tract. The pharmaceutical compositions may be formulated in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be formulated as a dry powder for insufflation, alone or in combination with an inert carrier such as lactose or phospholipids; and nasal drops. For intranasal use, the powder may comprise a bioadhesive agent, including chitosan or cyclodextrin.

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route.

Modified release dosage forms include delayed-, extended-, prolonged-, sustained-, pulsatile-, controlled-, accelerated- and fast-, targeted-programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

The pharmaceutical compositions disclosed herein in a modified release dosage form may be prepared by methods known to those skilled in the art, including direct compression, dry or wet granulation followed by compression, melt-granulation followed by compression.

Generally, the amount of an oligosaccharide disclosed herein required to be administered to the person can vary depending upon factors such as the risk and condition severity, the age of the person, the form of the composition, and other medications being administered to the person. It would be expected that an oligosaccharide described herein should be well tolerated irrespective of the age and condition of the subject. The dosage of oligosaccharide to be administered can readily be set by a medical practitioner and would generally be in the range from about 10 mg to about 20 g per day, in certain embodiments from about 10 mg to about 15 g per day, from about 100 mg to about 10 g per day, in certain embodiments from about 500 mg to about 15 g per day, in certain embodiments from about 1 g to about 7.5 g per day. An appropriate dose can be determined based on several factors, including, for example, the body weight and/or condition of the patient being treated, the severity of the condition, being treated, other ailments and/or diseases of the person, the incidence and/or severity of side effects and the manner of administration. Appropriate dose ranges can be determined by methods known to those skilled in the art. During an initial treatment phase, the dosing can be higher (for example 200 mg to 20 g per day, preferably 500 mg to 15 g per day, more preferably 1 g to 10 g per day, in certain embodiments 2.5 g to 7.5 g per day). During a maintenance phase, the dosing can be reduced (for example, 10 mg to 10 g per day, preferably 100 mg to 7.5 g per day, more preferably 500 mg to 5 g per day, in certain embodiments 1 g to 2.5 g per day).

Depending on the disorder to be treated and the injection in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.01 to about 2 grams, from about 0.05 to about 1 gram, or from about 10 to about 500 milligrams active ingredient(s) per dosage unit.

In certain embodiments, an appropriate dosage level is about 0.01 to about 5 g/kg patient body weight per day (mg/kg per day), about 0.01 to about 1 g/kg per day, about 0.01 to about 0.5 g/kg per day, or about 0.1 to about 500 mg/kg per day, which may be administered in single or multiple doses. A suitable dosage level may be about 0.1 to about 500 mg/kg per day, about 0.1 to about 250 mg/kg per day, or about 0.1 to about 100 mg/kg per day. Within this range the dosage may be about 0.01 to about 0.1, about 0.1 to about 1.0, about 1.0 to about 10, or about 10 to about 100 mg/kg per day.

The oligosaccharides disclosed herein may also be combined or used in combination with other agents useful in the treatment, prevention, or amelioration of one or more symptoms of an autoimmune disorder and/or inflammatory disorder. Or, by way of example only, the therapeutic effectiveness of one of the oligosaccharides described herein may be enhanced by administration of an adjuvant (i.e., by itself the adjuvant may only have minimal therapeutic benefit, but in combination with another therapeutic agent, the overall therapeutic benefit to the patient is enhanced). Additional therapeutic agents can include probiotics, prebiotics, and products administered to improve gastrointestinal health.

Such other agents, adjuvants, or drugs, may be administered, by a route and in an amount commonly used therefore, simultaneously or sequentially with an oligosaccharide as disclosed herein. When an oligosaccharide as disclosed herein is used contemporaneously with one or more other drugs, a pharmaceutical composition containing such other drugs in addition to an oligosaccharide disclosed herein may be utilized, but is not required. Accordingly, the pharmaceutical compositions disclosed herein include those that also contain one or more other active ingredients or therapeutic agents, in addition to an oligosaccharide disclosed herein.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. For example, the container(s) can comprise one or more oligosaccharides described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an oligosaccharide with an identifying description or label or instructions relating to its use in the methods described herein. In certain embodiments, a container comprises 2′FL.

A kit will typically comprise one or more additional containers, each with one or more of various materials (such as reagents, optionally in concentrated form, and/or devices) desirable from a commercial and user standpoint for use of an oligosaccharide described herein. Non-limiting examples of such materials include, but are not limited to, buffers, diluents, filters, needles, syringes; carrier, package, container, vial and/or tube labels listing contents and/or instructions for use, and package insert with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

### Example 1: 2′-FL Upregulates CEBPB Expression in LPS-Stimulated THP-1 Cells

THP-1 Cells were treated with 10 ng/mL LPS or 10 ng/mL LPS and 250 μM 2′FL and extracted for nascent RNAs at 30 min and 24 hours. The nascent RNAs were sequenced and analyzed as described in Azofeifa et al. (2018), Genome Res. 28: 334-344; the contents of which are expressly incorporated by reference herein.

When comparing the nascent RNAs of the 2′FL+LPS treated cells versus the LPS alone, CEBPB was enhanced and upregulated at both the 30 min and 24 hour time points, with a maximum enrichment score of 40 and 20, respectively.

### Example 2: Use of 2′-FL in Models of Frailty

The effectiveness of fucosylated oligosaccharides, such as 2′-FL, in improving frailty is assessed using one or more animal models of frailty. Such models have been described, for example, in Kane et al. (2016), Clin Interv Aging 11: 1519-1529; the contents of which are expressly incorporated by reference herein.

2′-FL can be assessed in the interleukin-10 homozygous knock out (IL-10^{tm/tm}) mouse model of frailty as described by Walston et al. (2002), Arch Intern Med. 2002; 162(20):2333-2341 and Ko et al. (2012), Age (Dodr): 34(3):705-715; the contents of each of which are expressly incorporated by reference herein. IL-10^{tm/tm} have a frail phenotype including inflammation and decreased muscle strength. IL-10^{tm/tm} mice are administered 2′-FL or placebo/vehicle and an improvement in strength, activity, weight, serum IL-6 level and/or skeletal muscle gene expression is assessed in 2′-FL-treated mice as compared to the placebo treated group.

The effect of 2′-FL can be assessed using the frailty assessment tool in mice as described in Parks et al. (2012), J Gerontol A Biol Sci Med Sci.: 67(3):217-227 and Graber et al. (2015), Biol Sci Med Sci.: 70(9):1045-1058; the contents of each of which are expressly incorporated by reference herein. 2′-FL and placebo/vehicle can be administered to male and female C₅₇BL/6 mice and frailty index (FI) score is calculated by determining the number of observed deficits, divided by the total number of possible deficits.

The effect of 2′-FL can be assessed in mouse frailty phenotype assessment models including, for example, the neuromuscular healthspan-scoring system (NMHSS) in male C57BL/6 mice or the mouse frailty assessment tool as described in Graber et al. (2013), Biol Sci Med Sci 68(11): 1326-1336 and Liu et al. (2013), Biol. Sci. Med Sci. 69(12): 1485-1489, the contents of which are expressly incorporated by reference herein. 2′-FL and placebo/vehicle is administered to mice and the score is calculated using the summed scores of a mouse's performance in the rotarod and inverted-cling grip tests, plus an in vitro muscle contractility assessment or calculated by measuring grip strength (assessed with the inverted-cling grip test), maximal walking speed (assessed by rotarod), physical activity (assessed by voluntary wheel running), and endurance (composite score of rotarod and grip strength performance).

While this invention has been particularly shown and described with references to preferred embodiments thereof, it will be understood by those skilled in the art that various changes in form and details may be made therein without departing from the scope of the invention encompassed by the appended claims.

1. A method of improving muscle strength or mobility in a subject in need thereof comprising administering to said subject an effective amount of 2′-fucosyllactose (2′FL).

2. The method of claim 1, wherein the effective amount is an amount that increases the expression of CEBPB.

3. The method of claim 1, wherein the subject is a human subject.

4. The method of claim 3, wherein the subject is suffering from low muscle mass or impaired physical mobility.

5. The method of claim 1, wherein the subject is suffering from and/or diagnosed with a condition selected from the group consisting of frailty or frailty syndrome, pre-frailty, sarcopenia, age-related muscle loss, metabolic syndrome, and a condition resulting in muscle atrophy and/or fatigue.

6. The method of claim 1, wherein the subject is suffering from and/or diagnosed with drug-induced muscle weakness or myopathy.

7. (canceled)

8. The method of claim 1, wherein the subject is suffering from muscle weakness associated with infection.

9. (canceled)

10. The method of claim 1, wherein the subject is suffering from muscle weakness associated with an endocrine condition, an inflammatory condition, a rheumatologic condition, an electrolyte syndrome, a neuromuscular condition, a neurological condition, or a genetic condition.

11. (canceled)

12. (canceled)

13. (canceled)

14. (canceled)

15. (canceled)

16. (canceled)

17. (canceled)

18. The method of claim 4, wherein the impaired physical mobility is due to stroke, rheumatoid arthritis, osteoarthritis, osteoporosis, osteopenia, limb fracture, multiple sclerosis, trauma, sepsis, or obesity.

19. The method of claim 4, wherein the impaired mobility is due to aging.

20. The method of and claim 2, wherein the subject has reduced CEBPB expression level prior to the initiation of the treatment, wherein the reduced CEBPB expression level is reduced as compared to a control expression level.

21. The method of claim 20, wherein the expression level is detected by measuring RNA.

22. The method of claim 20, the method further comprising obtaining a biological sample from the subject prior to the initiation of the treatment, detecting the expression level of CEBPB in the biological sample, and determining that the expression level of CEBPB is lower than that of the control expression level.

23. The method of claim 1, wherein the expression level of CEBPB increases after the initiation of the therapy.

24. The method of claim 1, wherein the subject is an adult.

25. (canceled)

26. (canceled)

27. The method of claim 1, wherein the treatment improves the subject's muscle strength or mobility as measured by lower handgrip muscle strength, short physical performance battery (SPPB) score, Timed Up and Go test (TUGT), walking speed (WS), and/or grip strength (GS).

28. The method of claim 1, wherein the 2′FL is administered orally.

29. The method of claim 1, wherein the amount of 2′FL is from 1 g to about 20 g per day.

30. The method of claim 1, wherein the 2′FL is administered in a pharmaceutical composition comprising a pharmaceutically acceptable carrier or excipients, wherein the pharmaceutical composition is not mammalian milk.

31. A method of increasing CEPBP expression in a subject in need thereof comprising administering to said subject an effective amount of 2′-fucosyllactose (2′FL).

32. The method of claim 31, wherein the effective amount is an amount that increases the expression of CEBPB.

33. The method of claim 31, wherein the subject is a human subject.

34. The method of claim 31, wherein the subject is suffering from low muscle mass or impaired physical mobility.

35. The method of claim 31, wherein the subject is suffering from and/or diagnosed with a condition selected from the group consisting of frailty or frailty syndrome, pre-frailty, sarcopenia, age-related muscle loss, metabolic syndrome, and a condition resulting in muscle atrophy and/or fatigue.

36. The method of claim 31, wherein the subject is suffering from and/or diagnosed with drug-induced muscle weakness or myopathy.

37. (canceled)

38. The method of claim 31, wherein the subject is suffering from and/or diagnosed with muscle weakness associated with infection.

39. (canceled)

40. The method of claim 31, wherein the subject is suffering from muscle weakness associated with an endocrine condition, an inflammatory condition, a rheumatologic conditions, an electrolyte syndrome, a neuromuscular condition, a neurological condition, or a genetic condition.

41. (canceled)

42. (canceled)

43. (canceled)

44. (canceled)

45. (canceled)

46. The method of claim 35, wherein the age-related muscle loss is at least partially due to diabetes or obesity.

47. (canceled)

48. (canceled)

49. (canceled)

50. The method of claim 31, wherein the subject is at risk of impaired physical mobility, muscle loss, or muscle weakness.

51. (canceled)

52. (canceled)

53. (canceled)

54. (canceled)

55. The method of and claim 31, wherein the subject has reduced CEBPB expression as compared to a control expression level prior to the initiation of the treatment.

56. The method of claim 55, wherein the expression level is detected by measuring RNA.

57. The method of claim 55, comprising obtaining a biological sample from the subject prior to the initiation of the therapy, detecting the expression level of CEBPB in the biological sample, and determining that the expression level of CEBPB is lower than that of the control express level prior to the initiation of the treatment.

58. The method of claim 31, wherein the expression level of CEBPB increases after the initiation of the therapy.

59. The method of claim 1, wherein the subject is an adult.

60. (canceled)

61. The method of claim 31, wherein the treatment improves the subject's muscle strength or mobility as measured by lower handgrip muscle strength, short physical performance battery (SPPB) score, Timed Up and Go test (TUGT), walking speed (WS), and/or grip strength (GS).

62. The method of claim 31, wherein the 2′FL is administered orally.

63. The method of claim 31, wherein the amount of 2′FL is from 1 g to about 20 g per day.

64. The method of claim 31, wherein the 2′FL is administered in a pharmaceutical composition comprising a pharmaceutically acceptable carrier or excipients, wherein the pharmaceutical composition is not mammalian milk.
